(19) United States
(12) Patent Application Publication
(10) Pub. No.: US 2013/0123719 A1
(43) Pub. Date: May 16, 2013

(54) MEDICATION COMPLIANCE PATCH AND CONTROL UNIT

(71) Applicants: Wei Mao, Palo Alto, CA (US); Henning Hayn, Stuttgart (DE); Jelena Madic, South San Francisco, CA (US); Ralf Schellin, Baden-Wurttemberg (DE)

(72) Inventors: Wei Mao, Palo Alto, CA (US); Henning Hayn, Stuttgart (DE); Jelena Madic, South San Francisco, CA (US); Ralf Schellin, Baden-Wurttemberg (DE)

(73) Assignee: Robert Bosch GmbH, Stuttgart (DE)

(21) Appl. No.: 13/295,044

(22) Filed: Nov. 12, 2011

(51) Int. Cl. A61M 35/00

(52) U.S. Cl. 604/304

(57) ABSTRACT

A medication patch compliance control unit system and method is disclosed. The control unit can include an attachment sensor, a user interface and a controller that receives signals from the attachment sensor and sends signals to the user interface. The control unit can include an attachment sensor, a wireless transceiver and a controller that receives signals from the attachment sensor and sends signals through the transceiver. The attachment sensor can be a temperature sensor, and the controller can determine attachment based on whether the sensor is reading temperatures in the normal body temperature range. The user interface can include visual and/or audible indicators. The control unit can include an expiration timer, and provide an expiration signal when the patch is expired. The transceiver can transmit medication patch data to a healthcare provider. The controller can record when the medication patch is attached to and detached from the patient.

### BACKGROUND OF THE INVENTION

This patent relates to medication compliance monitoring systems and techniques, and more particularly to a medication patch that monitors patch activity.

Medication compliance is an important issue for healthcare providers as well as patients themselves. Medications are usually most effective when taken according to particular time and dosage instructions to ensure proper treatment levels and to avoid potential side effects. However, roughly half of all prescription drugs to be taken for a prescribed period are either not taken for the complete period, or the prescriptions are never filled in the first place. This results in subsequent emergency hospitalizations and possible disputes of medical malpractice.

From a patient's perspective, there are at least two major scenarios that cause medication noncompliance. One scenario is that the patient is willing to and plans to take the medication but fails to follow the time and dosage instructions due to various limitations, for example forgetfulness, confusion by instructions, or physical inability to take the drug as prescribed. The other scenario is that the patient simply does not plan to take the medication or does not follow the time and dosage instructions intentionally, which can result in an inadequate amount of the medication being taken or an overdose of the medication. The reasons behind the patient noncompliance can include disbelief that the drug is necessary or is helping, feeling that the prescribed dosage is too low, fear of side effects, experiencing actual side effects, belief that symptoms have disappeared so the drug is no longer needed, and desire to save money.

It would be desirable to have a device and/or method to help improve or record a patient's medication compliance activity. Such a device and/or method could help in many ways. For example, it could help a patient adhere to the instructions better if the patient plans to take the medication. Alternatively, it could record the fact that the medication was never taken or that an incorrect amount was taken to alleviate any potential disputes over prescription errors.

### SUMMARY OF THE INVENTION

The medication compliance patch utilizes miniaturized electronics that can provide various benefits. For example, the medication compliance patch can be configured to detect and monitor skin contact time of the patch, or to give guidance to a patient to better abide by medication instructions, or to help a healthcare provider monitor a patient's medication usage, or to perform various other features disclosed herein.

An embodiment of a medication compliance control unit for integration into a medication patch is disclosed. The medication patch includes an adhesive area for attachment of the patch to a patient and a medicated area containing medication for application to the patient. The medication compliance control unit includes an attachment sensor, a user interface and a controller. The attachment sensor detects attachment of the medication patch to the patient. The user interface is used to communicate information to the patient. The controller receives signals from the attachment sensor and sends signals to the user interface, and at least some of the signals sent to the user interface are based on the received signals from the attachment sensor. The attachment sensor can be a temperature sensor, and the controller can determine attachment to the patient based on whether the temperature sensor is reading temperatures in the normal range of body temperatures. The user interface can include a visual indicator that displays a first color when the attachment sensor detects attachment to the patient and displays a second color when the attachment sensor no longer detects attachment to the patient. The user interface can include an audible indicator that transmits a warning signal when the attachment sensor detects detachment of the medication patch from the patient. The medication compliance control unit can include an expiration timer, and the controller can provide an expiration signal through the user interface when the expiration timer indicates that the medication patch is expired.

The medication compliance control unit can also include a wireless transmitter that transmits data pertaining to the medication patch. The controller can record when the medication patch is attached to the patient and when the medication patch is detached from the patient. The wireless transmitter can transmit when the medication patch is attached to the patient and when the medication patch is detached from the patient. The wireless transmitter can transmit a notification signal when the medication patch has not been worn for a predetermined period of time.

An alternative embodiment of a medication compliance control unit for integration into a medication patch is disclosed that includes an attachment sensor, a wireless transceiver and a controller. The attachment sensor detects attachment of the medication patch to the patient. The wireless transceiver communicates data pertaining to the medication patch. The controller receives signals from the attachment sensor and sends signals through the wireless transceiver. The controller can send data to a healthcare provider through the wireless transceiver. The attachment sensor can detect both attachment and detachment of the medication patch to/from the patient; and the controller can send attachment and detachment notifications through the wireless transceiver based on the signals from the attachment sensor.

A method is disclosed for controlling a medication patch that includes a medication compliance control unit. The method includes detecting attachment of the medication patch to a patient, detecting detachment of the medication patch from the patient, and signaling expiration of the medication patch. Detecting attachment of the medication patch to the patient can include sensing body temperature measurements using a temperature sensor, starting an expiration timer, and recording an ON event. Detecting detachment of the medication patch from the patient can include, after detecting attachment of the medication patch and prior to expiration of the medication patch, no longer sensing body temperature measurements using the temperature sensor, suspending the expiration timer, recording an OFF event, and signaling detachment of the medication patch through a user interface. Signaling expiration of the medication patch can include detecting when the expiration timer reaches an expiration value, recording a time expired event, and activating an expiration signal through the user interface.

The method for controlling a medication patch that includes a medication compliance control unit can also include detecting reattachment of the medication patch to the patient. Detecting reattachment of the medication patch to the patient can include, after detecting detachment of the medication patch and prior to expiration of the medication patch, again sensing body temperature measurements using the temperature sensor, resuming the expiration timer, recording an additional on event, and signaling attachment of the medication patch through the user interface.

The method for controlling a medication patch that includes a medication compliance control unit can also include indicating a previously expired medication patch by performing the following steps after signaling expiration of the medication patch: sensing body temperature measurements using a temperature sensor, and activating an expiration signal through a user interface.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 illustrates an exemplary embodiment of a medication compliance patch; and

FIG. 2 illustrates an exemplary control flow for an embodiment of a medication compliance patch.

Corresponding reference characters indicate corresponding parts throughout the several views. Although the exemplification set out herein illustrates embodiments of the invention, in several forms, the embodiments disclosed below are not intended to be exhaustive or to be construed as limiting the scope of the invention to the precise forms disclosed.

### DESCRIPTION OF EXEMPLARY EMBODIMENTS

One category of medications is in the form of wearable patches, where the medications are embedded in disposable material attached to skin with adhesives and are absorbed by the skin gradually. Examples include, but are not limited to, nicotine patches, pregnancy patches, and pain medication patches. Such medication patches generally need to be worn over a certain period of time. As the medications wear out, the patch needs to be replaced.

A medication compliance patch can be configured to detect whether the patch is being worn, to record the duration over which the patch is being worn, to indicate visually and/or audibly when the patch needs to be replaced, or to transmit relevant information to a central location for review by a healthcare provider. If the patient is actively using a medication patch, it can also remind the patient to replace the patch at the right time. If the patch is removed prematurely or was never worn, it can record that information and possibly send a notification to a healthcare provider.

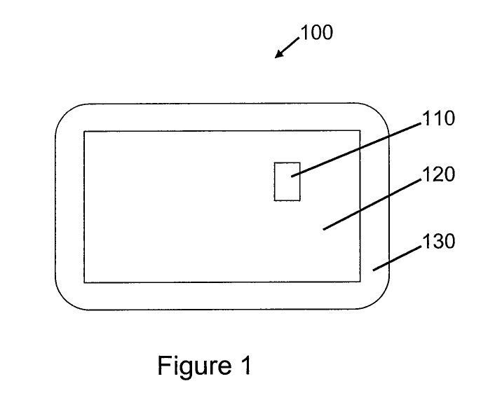

FIG. 1 shows an exemplary embodiment of a medication compliance patch 100. The medication compliance patch includes a compliance control unit 110, a medicated area 120 and an adhesive area 130 for attaching the patch 100 to a patient. The compliance control unit 110 can have contact with the patient's skin or be in close proximity to the skin.

The compliance control unit 110 can detect and monitor the usage or activity of the patch 100. The compliance control unit 110 can include one or more of the following components:

temperature sensor,

low power microprocessor,

user interface,

battery,

other sensors,

wireless transceiver.

The user interface can include, for example, an indicator light (for example, a light emitting diode (LED)), a speaker, a display, a pushbutton, etc. Other sensors can include, for example, ambient light sensors, accelerometers, gyroscopes, compasses, pressure sensors, optical sensors, biochemical sensors, etc.

A temperature sensor can detect skin contact by detecting normal human body temperature. A low power microprocessor can be triggered ON if skin contact is detected or the accelerometer detects movement. The low power microprocessor can be triggered OFF if skin contact is not detected. The low power microprocessor can alternatively wait until data recording and transmission are completed before powering OFF after skin contact is no longer detected. A user interface can communicate warning messages or reminders to a patient, for example “time to change patch.” Optional sensors can serve as alternatives or be combined together with the temperature sensor to detect skin contact or other vital signs of the patient or the patch. A wireless transceiver can transmit recorded data, such as patch activities or the absence of patch activities, to healthcare providers. Data transmission can be performed by various methods known in the art, for example through a dedicated hub (e.g., Bluetooth or WiFi at home or at a hospital), or over the air directly (e.g, a 3G network).

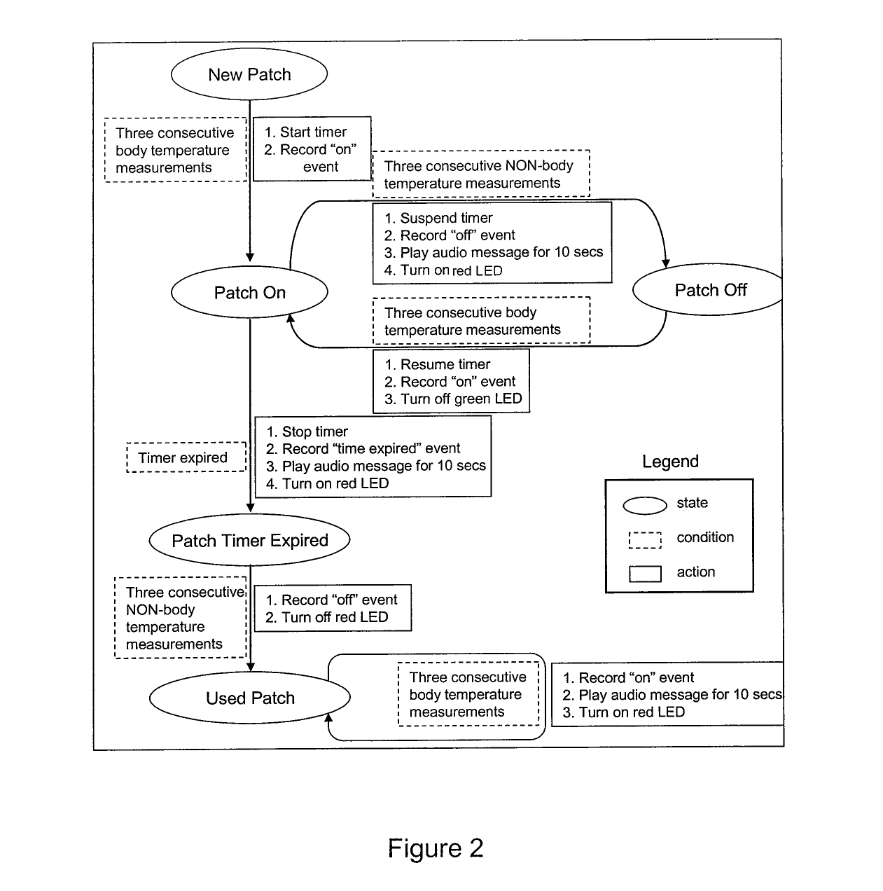

An exemplary control diagram for a medication compliance patch is shown in FIG. 2. For this embodiment, the patch 100 includes a compliance control unit 110 with at least a temperature sensor, a low power microprocessor and a user interface. A new patch 100 can initially be inactive in a “new patch” state awaiting placement on a patient's skin. The temperature sensor can be used to detect placement on the patient's skin. In this embodiment, when the temperature sensor senses values that are within the normal human body temperature range consistently over three samples, a timer is started and an “ON” event or “skin contact” event is recorded with a time stamp by the compliance control unit 110, and the patch 100 transitions to a “patch ON” state. If there are only one or two isolated readings consistent with body temperature, then the readings can be discarded as noise. If the patch has not been worn within an instructed amount of time, the user interface can warn the patient via visual signals and/or audio messages or tones.

In the “patch ON” state, the temperature sensor continues to monitor temperature. If the temperature sensor returns three values outside the body temperature range, the compliance control unit 110 can record an “OFF” event with a time stamp. When an “OFF” event is recorded, the patch 100 transitions from the “patch ON” state to the “patch OFF” state. The “OFF event” can also include suspending the timer, and activating visual and/or audio signals in the user interface. The user interface signals can warn the patient or healthcare staff that the patch is not in contact with the patient's skin.

In the “patch OFF” state, if the patch is put back on before the power is exhausted, the patch can transition back to the “patch ON” state. The patch 100 could be removed temporarily for various reasons. For example, the patient might find the patch area to be itchy and may decide to move it to a different area on the body. In this embodiment, if the patch 100 senses three consecutive body temperature measurements, the patch transitions back to the “patch ON” state and records an “ON” event with a time stamp. When transitioning back to the “patch ON” state, the compliance control unit 110 also resumes the active timer and turns OFF the warning signals of the user interface.

A timer expiration event can be used to determine when it is time to change the patch 100. When the timer expires, the patch transitions to the “patch timer expired” state where the compliance control unit 110 can stop the timer, record a “time expired” event and provide audio and/or visual signals through the user interface. Different notification signals can be used to remind the patient to change the patch. For example, a green LED can indicate that the patch 100 is ON and a red LED can indicate that the patch 100 is OFF or expired.

From the “patch timer expired” state when the compliance control unit senses that the patch has been removed, for example by no longer sensing body temperature, the patch can transition to a “used patch” state. When transitioning to the “used patch” state, the compliance control unit 110 can record an “OFF” event and turn OFF the signals indicating that the timer expired.

In the “used patch” state, when the compliance control unit senses that the patch has been reattached to the skin, for example by sensing body temperature again, the compliance control unit 110 can record an “ON” event and activate user interface warning signals to indicate that the patch has already been used. The compliance control unit 110 can continue to provide the warnings until the patch is removed or power is exhausted, and the patch remains in the “used patch” state.

The compliance control unit 110 can be used as a standalone device with wireless connections to help patients comply with the medication instructions. With a wireless radio on board, the compliance control unit 110 can communicate the acquired data such as patch activities or the absence of patch activities over an extended period to healthcare providers. A patch identifier can be transmitted along with the acquired data. The patch identifier can be a unique identifier for the particular medication patch, containing information regarding the medication type and dosage. This can enable the healthcare providers to monitor an individual patient's medication compliance. It also can enable the healthcare provider to detect events where multiple patches are worn simultaneously by the same patient to prevent overdose.

While this invention has been described as having an exemplary design, the present invention may be further modified within the spirit and scope of this disclosure. This application is therefore intended to cover any variations, uses, or adaptations of the invention using its general principles.

We claim:

1. A medication compliance control unit for integration into a medication patch that includes an adhesive area for attachment of the patch to a patient and a medicated area containing medication for application to the patient, the medication compliance control unit comprising:
an attachment sensor detecting attachment of the medication patch to the patient;
a user interface communicating information to the patient; and
a controller receiving signals from the attachment sensor and sending signals to the user interface, at least some of the signals sent to the user interface being based on the received signals from the attachment sensor.

2. The medication compliance control unit of claim 1, wherein the attachment sensor is a temperature sensor; and the controller determines attachment to the patient based on whether the temperature sensor is reading temperatures in the normal range of body temperatures.

3. The medication compliance control unit of claim 1, wherein the user interface includes a visual indicator controlled by the controller, the visual indicator displaying a first color when the attachment sensor detects attachment to the patient and displaying a second color when the attachment sensor no longer detects attachment to the patient.

4. The medication compliance control unit of claim 1, wherein the user interface includes an audible indicator controlled by the controller, the audible indicator transmitting a warning signal when the attachment sensor detects detachment of the medication patch from the patient.

5. The medication compliance control unit of claim 1, wherein the controller records when the medication patch is attached to the patient and when the medication patch is detached from the patient.

6. The medication compliance control unit of claim 5, further comprising a wireless transmitter controlled by the controller, wherein the controller transmits through the wireless transmitter in response to the medication patch being attached to the patient and in response to the medication patch being detached from the patient.

7. The medication compliance control unit of claim 1, further comprising a wireless transmitter controlled by the controller, wherein the controller transmits data pertaining to the medication patch through the wireless transmitter.

8. The medication compliance control unit of claim 7, wherein the controller transmits a notification signal through the wireless transmitter in response to the medication patch having not been worn for a predetermined period of time.

9. The medication compliance control unit of claim 1, further comprising an expiration timer, the controller providing an expiration signal through the user interface in response to the expiration timer indicating that the medication patch is expired.

10. A medication compliance control unit for integration into a medication patch that includes an adhesive area for attachment of the patch to a patient and a medicated area containing medication for application to the patient, the medication compliance control unit comprising:
an attachment sensor detecting attachment of the medication patch to the patient;
a wireless transceiver communicating data pertaining to the medication patch; and
a controller receiving signals from the attachment sensor and sending signals through the wireless transceiver.

11. The medication compliance control unit of claim 10, wherein the controller sends data to a healthcare provider through the wireless transceiver.

12. The medication compliance control unit of claim 11, wherein the attachment sensor also detects detachment of the medication patch from the patient; and the controller sends attachment and detachment notifications through the wireless transceiver based on the signals from the attachment sensor.

13. A method of controlling a medication patch that includes a medication compliance control unit, the method comprising:
detecting attachment of the medication patch to a patient;
detecting detachment of the medication patch from the patient; and
signaling expiration of the medication patch.

14. The method of claim 13, wherein detecting attachment of the medication patch to the patient comprises sensing body temperature measurements using a temperature sensor.

15. The method of claim 14, wherein detecting attachment of the medication patch to the patient further comprises:
starting an expiration timer, and
recording an ON event.

16. The method of claim 15, wherein detecting detachment of the medication patch from the patient comprises the following steps after detecting attachment of the medication patch and prior to expiration of the medication patch:
sensing absence of body temperature measurements using the temperature sensor;
suspending the expiration timer,
recording an OFF event; and
signaling detachment of the medication patch through a user interface.

17. The method of claim 16, wherein signaling expiration of the medication patch comprises:
detecting when the expiration timer reaches an expiration value.
recording a time expired event; and
activating an expiration signal through the user interface.

18. The method of claim 16, further comprising:
detecting reattachment of the medication patch to the patient;

19. The method of claim 18, wherein detecting reattachment of the medication patch to the patient comprises the following steps after detecting detachment of the medication patch and prior to expiration of the medication patch:
sensing body temperature measurements again using the temperature sensor;
resuming the expiration timer,
recording an additional ON event; and
signaling attachment of the medication patch through the user interface.

20. The method of claim 13, further comprising indicating a previously expired medication patch by performing the following steps after signaling expiration of the medication patch:
sensing body temperature measurements using a temperature sensor; and
activating an expiration signal through a user interface.
